(19) United States
(12) Patent Application Publication
(10) Pub. No.: US 2003/0119109 A1
(43) Pub. Date: Jun. 26, 2003

(54) Efficient 13C/15N double labeling of proteins in a methanol-utilizing strain (Mut+) of pichia pastoris

(76) Inventors: Harrold Alfred van den Burg, Wageningen (NL); Jacobus Johannes Marie Vervoort, Bennekom (NL); Peter Jozef Gerard Marie De Wit, Rhenen (NL)

Correspondence Address: ROTHWELL, FIGG, ERNST & MANBECK, P.C., 1425 K STREET, N.W., SUITE 800, WASHINGTON, DC, 20005, US

(21) Appl. No.: 10/205,616

(22) Filed: Jul. 26, 2002

Related U.S. Application Data
(60) Provisional application No. 60/307,610, filed on Jul. 26, 2001.

(51) Int. Cl. C12P 21/02; C12N 1/18

(52) U.S. Cl. 435/69.1; 435/254.23

(57) ABSTRACT

Cost effective ¹³C/¹⁵N-isotope labeling of the avirulence protein AVR4 (10 kDa) of the fungal tomato pathogen Cladosporium fulvum was achieved with the methylotrophic yeast Pichia pastoris in a fermentor. The ¹³C/¹⁵N-labeled AVR4 protein accumulated to 30 mg/L within 48 h in an initial fermentation volume of only 300 mL, while prolonged optimized overexpressions yielded 126 mg/L. These protein yields were 24 fold higher in a fermentor than in flask cultures. In order to achieve these protein expression levels, we used the methanol-utilizing strain (Mut⁺) of Pichia pastoris which has a high growth rate while growing on methanol as only carbon source. In contrast, the methanol-sensitive strain (Mut^{S}) could intrinsically yield comparable protein expression levels, but at the dispense of additional carbon sources. Although both strains are generally used for heterologous protein expression, we show that the costs for ¹³C-isotope labeling can be substantially reduced using the Mut⁺ strain compared to the Mut^{S} strain, as no ¹³C₃-glycerol is required during the methanol-induction phase. Finally, nitrogen limitations were precluded for ¹⁵N-labeling by an optimal supply of 10 g/L (¹⁵NH₄)₂SO₄ every 24 h.

This application claims priority from copending provisional application serial No. 60/307,610, filed Jul. 26, 2001.

### BACKGROUND OF THE INVENTION

1. Technical Field

The invention relates to the field of yeast fermentation. In particular, the invention provides a method for efficient production of isotopically labeled proteins using Pichia pastoris.

2. Description of the Background Art

For the detailed analysis of proteins, structural biologists often require advanced eukaryotic heterologous expression systems as many of the proteins under study require extensive posttranslational modifications for correct folding to the native state. The methylotrophic yeast Pichia pastoris has been refined into a host in which protein secretion and posftranslational modifications are easily accomplished and has, therefore, gained much attention as a heterologous expression system recently (Cregg et al., 1987; Tschopp et al., 1987; Cregg et al., 1993; Romanos, 1995; Sreekrishna et al., 1997).

For heteronuclear NMR experiments proteins need to be enriched with ¹⁵N- or ¹³C/¹⁵N-isotopes. To a large extent, Escherichia coli has been the host-of-choice for isotope labeling of proteins, as simple media for flask cultures are well-defined and rather inexpensive. However, the yeast P. pastoris has scarcely been used for ¹³C/¹⁵N-isotope labeling of proteins as no cost-effective protocol for routine isotope labeling has been available so far. ¹³C/¹⁵N-isotope labeling in P. pastoris has exclusively been carried out in flask cultures for highly expressed proteins (Laroche et al., 1994; Abbate et al., 1999; Morgan et al., 1999). Even ¹⁵N-labeling has almost exclusively been obtained in flask cultures (Denton et al., 1998; McAlister et al., 1998; Mine et al., 1999). Yet, the advantages of P. pastoris are best exploited in a fermentation with its intrinsically high growth rate and high cell density by which protein yields increase 20-100 fold (Stratton et al., 1998; Wood and Komives, 1999).

Optimized fermentation protocols resulting in high cell densities for P. pastoris have been well documented (Stratton et al., 1998; Wood and Komives, 1999). Briefly, a fermentation consists of three growth phases i.e. a glycerol batch phase, a glycerol fed-batch phase, and a methanol-induction phase. In the first phase, the methanol inducible alcohol oxidase 1 (AOX1) promoter controlling the heterologous gene is completely repressed by an excess of glycerol. After this initial glycerol supply has been depleted, a minimal glycerol feed for a few hours ensures the derepression of the AOX1 promoter and a smooth transition to the third phase in which methanol is added as inducer. In the first two phases the biomass accumulates rapidly, while in the last phase the heterologous protein is expressed at high levels.

The growth characteristics in the methanol-induction phase depend on the phenotype of the P. pastoris transformants. When the AOX1 gene is intact (Mut⁺ strain), the transformant will grow at wild-type rate on methanol. However, when the AOX1 gene is disrupted (Mut^{S} strain), the methanol metabolism will rely on the less active AOX2 gene (Clare et al., 1991; Digan et al., 1989). Consequently, the Mut^{S} strain has an intrinsic lower growth rate on methanol than the Mut⁺ strain. The fermentation run with the Mut^{S} strain needs, therefore, to be extended for comparable protein expression levels. The growth rate of the Mut^{S} strain will, however, increase if a combined feed of glycerol and methanol is applied in the methanol-induction phase. In conclusion, comparable expression levels can be obtained with the two strains if the proper fermentation protocol is used. (Brierley et al., 1990; Clare et al., 1991).

A fermentor offers the best options to reduce the costs for ¹³C-labeling of proteins in P. pastoris as high expression levels combined with the tight control of biomass accumulation allow the volume of the fermentor to be small compared to flask cultures without concessions to the yield. We used the Mut⁺ strain rather than the Mut^{S} strain for the fermentation for a few reasons. Firstly, the growth rate, which influences the protein expression levels, is significantly reduced for the Mut^{S} strain while growing on methanol alone. Secondly, addition of ¹³C₃-glycerol is relative expensive as ¹³C₃-glycerol is 3.5 times more expensive than ¹³C-methanol. Thirdly, the glycerol fed-batch phase could be shorter as the biomass continues to accumulate during the methanol-induction phase of the Mut⁺ strain. This glycerol fed-batch phase normally ensures the derepression of the AOX1 promoter and high biomass accumulation. The latter is of less importance as yields can be optimized for NMR purposes. And finally, when both grow on methanol alone, the total fermentation time is significantly shorter for the Mut⁺ strain than for the Mut^{S} strain.

Hereby, we successfully labeled the avirulence protein AVR4 of the fungus Cladosporium fulvum with the ¹³C-carbon and ¹⁵N-nitrogen isotopes in an initial volume of only 300 mL. To achieve ¹⁵N-labeling, we had to optimize the (¹⁵NH₄)₂SO₄ supply. At the expense of only a minimal amount ¹³C-carbon being consumed, the fermentation still yielded 30 mg/L of AVR4 protein. This general applicable protocol yielded enough AVR4 protein for at least two samples of 2 mM each for NMR measurements.

### SUMMARY OF THE INVENTION

The present invention is directed to a method for cost-effective production of isotopically labeled protein material suitable for study with NMR. Isotopic labels may include 13C, 15N or both 13C and 15N. Although the examples provided here generally refer to AVR4, the avirulence protein of the fungus Cladosporium fulvum, persons of skill in the art will readily see that the methods are applicable to any protein which the practitioner wishes to prepare for study in an isotopically labeled form. The method produces proteins in a properly folded and glycosylated form since the method involves fermentation with a eukaryote, the yeast Pichia pastoris. The yeast cells can be induced to overexpress the desired protein and using the inventive methods can produce isotopically substituted protein in a more cost-effective manner than has been achievable using prior art methods. The invention therefore is useful to produce any protein material with isotopic substitutions which enable structural studies of the protein by NMR in its natural conformation, with proper tertiary structure and glycosylations. Isotopic labeling can be achieved more cheaply by using the Mut⁺ strain of P. pastoris using the methods of the present invention, representing an improvement upon the prior art.

### BRIEF DESCRIPTION OF THE DRAWINGS

FIG. 1. Secretion of AVR4 (A and D), total secreted protein (B and E) and biomass (C and F), as determined by ELISA, Bradford assay, and CDW, respectively, are shown for the methanol induction phase. The amount of (NH₄)₂SO₄ supplied during the fermentations was optimized, as (NH₄)₂SO₄ limitations strongly affect the protein secretion levels. Different aliquots, i.e. 5 g (p), 10 g (l) en 20 g (NH₄)₂SO₄ (Δ), were added every 24 h after the start of the fermentation. The fermentation proceeded for at least 96 h to observe the effect of increased salt concentration on the growth of the yeast. The fermentations using (NH₄)₂SO₄ are compared to the optimal fermentation using NH₄OH (o). The ¹³C/¹⁵N-labeling was performed in an initial volume of only 300 mL medium with 10 g of (NH₄)₂SO₄ supplied every 24 h (Ü; D-F) and is compared to the original 1 liter fermentation (l; A-F) The glycerol fed-batch phase (second phase) was only 30 min in the ¹³C/¹⁵N labeling experiment, which explains the decreased CDW at the strart of the induction. The third phase for the ¹³C/¹⁵N labeling experiment only lasted for 48 h, during which 30 mg/L AVR4 had accumulated over this relatively short period.

FIG. 2. Secretion of the AVR4 protein in the medium is shown on Coomassie-brilliant-blue stained tricine SDS-PAGE gel. The numbers above each lane correspond to consecutive time points in the methanol induction phase. Samples were taken from the fermentation experiment in which 10 g (NH₄)₂SO₄ per liter was added every 24 h (FIG. 1;l). The AVR4 protein indicated by an arrow is the most abundant protein. Few other proteins are secreted. Molecular weight marker was loaded in the most right lane. 20 μL culture medium was loaded in each lane.

FIG. 3. Mass determination of the (A) ¹³C/¹⁵N-, (B) ¹⁵N-labeled and (C) unlabeled AVR4 protein as determined by MALDI-TOF mass spectrometry. The theoretical mass of AVR4 with 4 disulfide bonds is 9544.1 m/z. The mature AVR4 protein contains 110 nitrogen and 421 carbons atoms. The labeling efficiency is +98% for both ¹⁵N- and ¹³C/¹⁵N-labeled AVR4. Relative intensities are shown on the y-axis. The masses of the singly charged ions are indicated by their mass-over-charge (m/z) values. Masses of doubly charged ions are indicated by [M+2H]²⁺.

FIG. 4. NMR spectra of labeled AVR4: (A) ¹H-¹⁵N HSQC of the ¹⁵N-labeled AVR4 sample, (B) a ¹³C-decoupled ¹H-¹⁵N HSQC of the ¹³C/¹⁵N labeled AVR4 sample, and (C) a H(N)CO experiment of ¹³C/¹⁵N labeled AVR4. Conditions were as described in materials and methods.

### DETAILED DESCRIPTION OF THE PREFERRED EMBODIMENTS

Materials and Methods

Materials

Chemicals of the highest grade available were obtained from Merck. Yeast Nitrogen Base, and Maxisorp Nunc immuno plates were obtained via Life Technologies. Isotope enriched 98%+¹⁵N-ammonium sulfate was from Cambridge Isotope Laboratories Inc. (Andover, Mass.); 99%+¹³C-methanol and 99%+¹³C-glycerol were acquired via Cortec (Paris, France).

Methods

Expression levels of AVR4 were visualized by tricine SDS-PAGE (Schägger and Von Jagow, 1987). Necrosis-inducing activity of AVR4 in samples was routinely tested on Moneymaker Cf4 using Moneymaker Cf0 tomato plants as control (Joosten et al., 1994). Heterologous AVR4 was identified on Western blots with a polyclonal antibody raised in Rabbit against the AVR4 protein purified from apoplastic fluid of a compatible interaction between race 5 of C. fulvum and tomato genotype Cf5 (Joosten et al., 1997). Final purity of heterologous AVR4 was determined on an analytical, 25×4.6 mm, 300 Å pore, C18 Delta Pak RP-HPLC column (Waters). AVR4 concentrations were determined in a direct ELISA assay with an anti AVR4 polyclonal antibody raised in Chicken (IgY). Samples were coated in 100 mM sodium acetate buffer (pH 4.0), overnight at 8° C. and wells were blocked with 0.5% (w/v) Bovine Serum Albumine in 100 mM potassium phosphate buffer (pH 7.0), 150 mM sodium chloride at 37° C. for 2 h. The primary antibody was detected with alkaline phosphatase conjugated to a secondary Rabbit anti-Chicken IgG (Sigma). Total protein contents were determined using Bradford reagent (Sigma), and referenced to Bovine Serum Albumine. Mass determinations were performed with a delayed extraction MALDI-TOF mass spectrometer (Perseptive Biosystems, Framingham Mass.). The MALDI-TOF samples were applied in α-cyano-4-hydroxycinnamic acid (Sigma) as matrix using the dried droplet method (Karas and Hillenkamp, 1988; Kussmann et al., 1997). Spectra were averages of 100-256 consecutive laser pulses. The instrument is generally operated at an acceleration voltage of 22 kV combined with delayed extraction. Spectra were calibrated using bovine Cytochrome C (12,230.9 m/z), Bovine Insulin (5,734.6 m/z) and Microperoxidase 8 (MP8, 1,506.5 m/z; Primus et al., 1998).

Growth Media

The media BMGY, BMMY, MD, and YPD were as described in the Invitrogen manual (URL: http://www.invitrogen.com/manuals.html, version L). One liter of FM22 fermentation medium contained 42.9 g KH₂PO₄, 10.0 g (NH₄)₂SO₄, 1.0 g CaSO₄.2H₂O, 14.3 g K₂SO₄, 11.7 g MgSO₄.7H₂O, and 40 g glycerol (Laroche et al., 1994). The amount of (NH₄)₂SO₄ was increased compared to the original paper (see Results section for more details). The 42.9 g KH₂PO₄ and the 10.0 g (NH₄)₂SO₄ were both substituted by 26.7 mL 85% H₃PO₄ and 4.13 g KOH in the FM22 medium when NH₄OH was used for the pH control in the fermentor. Trace salt solution PMT4 consisted of 2.0 g CuSO₄.5H₂O, 0.08 g Nal, 3.0 g MnSO₄.H₂O, 0.2 g Na₂Mo₂O₄.2H₂O, 0.02 g H₃BO₃, 0.5 g CaSO₄.2H₂O, 0.5 g CoCl₂, 7.0 g ZnCl₂, 22 g FeSO₄.7H₂O, 0.2 g biotin, and 1 mL concentrated H₂SO₄ per liter. To one liter FM22 medium 2.5 mL of PMT4 is added prior to inoculation. To the methanol supply 4 mL PMT4/L was added.

Shake Flask Culture

P. pastoris GS115 His⁺/mut⁺ transformants transgenic for Avr4 were selected for production of the AVR4 protein in BMMY as described in the Invitrogen manual. A mutation coding for a Ser-to-Ala amino acid substitution was introduced in the Avr4 gene at the position of the potential N-glycosylation site. Growth of P. pastoris was performed in two liter baffled flasks containing 200 mL BMGY shaken at 250 rpm for 2 days at 30° C. After 24 h, medium was exchanged by centrifugation at 3,000 g for 5 min and subsequently, the heterologous gene was induced by resuspending the cells in BMMY medium. Growth continued for another 96 h with additional pulses of 1% (v/v) methanol every 24 h. Supernatant was collected by centrifugation at 3,000 g followed by 10,000 g (both for 15 min), and was stored at −70° C. till further purification.

High Cell Density Fermentation of P. pastoris (Used for Large-scale AVR4 Production)

Fermentation was performed according to the procedure described by Stratton et al. (1998). Starter cultures of 50 mL were grown for 2 days at 30° C. (OD₆₀₀>10), and were used to inoculate a 2 L vessel containing 900 mL FM22 medium. Starter cultures originated from a fresh colony grown on a MD or YPD plate. After autoclaving the pH was adjusted to 4.9 with 5.0 M KOH or 25% (w/v) NH₄OH. Agitation was kept at 1200 rpm, and the airflow was maintained at 1-2 vvm to keep the dissolved oxygen (DO) levels at least above 30%. If needed, excessive foaming was prevented by adding a few droplets of Antifoam 289 (Sigma). Approximately 20 h after inoculation, glycerol depletion was observed by a sharp increase of the DO. At this stage the glycerol-feed was started at a rate of 10 mL·L⁻¹·h⁻¹ (glycerol fed-batch phase), and properly adjusted to maintain a steady DO reading (near 35%). After 4 h, the methanol feed was started at a rate of 3.4 mL·L⁻¹·h⁻¹. The methanol feed rate was step-wise increased to 6 mL·L⁻¹·h⁻¹ as soon as the culture had fully adapted to growth on methanol (4-6 h). To prevent methanol accumulation a DO spike was performed (a sharp increase in the DO levels has to occur after a halt of the methanol supply; Stratton et al., 1998).

Isotope Labeling in the Fermentor with ¹⁵N-ammonium Sulfate, ¹³C-glycerol, and ¹³C-methanol

All media contained isotope enriched substrate. A small culture of 300 mL FM22 medium was optimized for double labeling. The glycerol fed-batch phase lasted only for 30 min in the ¹³C/¹⁵N-labeling experiment. During this phase, a 50% (w/v) ¹³C₃-glycerol supply was fed at a rate of 0.2 mL/min (a total of 3 g ¹³C₃-glycerol was added). The ¹³C-methanol was diluted to 25% (w/v) to maintain a continuous methanol supply without toxic effects. The methanol-induction phase lasted 48 h (A total of 50 g of ¹³C-methanol was added). Aliquots of 10 g (¹⁵NH₄)₂SO₄/L were supplied every 24 h. After purification ¹³C/¹⁵N-isotope enrichment of AVR4 was determined by MALDI-TOF mass spectrometry and NMR spectroscopy.

Purification of AVR4 Protein

Cell free culture filtrate containing the AVR4 protein was brought to 45% (NH₄)₂SO₄ saturation and was stirred for 30 min at 4° C. followed by centrifugation at 13,000 g for 20 min The supernatant was applied at a rate of 5.0 mL/min to an equilibrated Phenyl Sepharose (high sub) Fast Flow column (26×200 mm, Amersham-Pharmacia). Prior to use the column had been equilibrated with 5 column volumes buffer A (10 mM Tris-HCl, pH 8.6, and 1 mM EDTA) and 2 column volumes buffer B (10 mM Tris-HCl, pH 8.6, 1 mM EDTA, and 45% saturated (NH₄)₂SO₄). The column was extensively washed with buffer B before eluting the AVR4 protein with a linear gradient of 300 mL going from buffer B to buffer A. Fractions were checked for AVR4 content by SDS-PAGE. The AVR4 containing fractions were pooled and were exhaustively dialyzed against buffer A. The desalted fractions were loaded at a flow rate of 2.0 mL/min on a Q-Sepharose Fast Flow column (16×240 mm, Amersham-Pharmacia). Prior to use the Q-Sepharose column had been equilibrated with buffer C (1.0 M NaCl, 10 mM Tris-HCl, pH 8.6, and 1 mM EDTA) followed by 5 volumes of buffer A. The flow through, which contained the AVR4 protein, was acidified with TFA and injected on a C4 RP-HPLC column (25×200 mm, 300 Å, Waters). The AVR4 protein eluted at 30% Acetonitril, 0.1% TFA. The collected AVR4 protein fraction was lyophilized prior to storage.

NMR Spectroscopy

The NMR samples in a Shigemi tube (Tokyo, Japan) contained 2.3 mM AVR4 protein dissolved in 90% H₂O/10% D₂O (v/v), 20 mM Acetate-d₄, and 50 mM NaCl at pH 4 in a total volume of 250 μl. NMR experiments were performed on a Bruker Avance 600 Mhz spectrometer equipped with a triple-resonance, pulsed-field gradient probe operating at a temperature of 25° C. Residual TFA was removed from the sample by extensive dialysis. ¹H chemical shifts were referenced to sodium 2,2-dimethyl-2-silapentane-5-sulfonate (DSS), ¹⁵N and ¹³C chemical shifts were indirectly referenced (Wishart et al., 1995; Markley et al., 1998). Heteronuclear sensitivity enhanced ¹H-¹⁵N HSQC spectra with pulsed-field gradients and WATERGATE (Kay et al., 1992; Piotto et al., 1992; Stonehouse et al., 1995), as well as HNCO spectra (Peelen et al., 1996) were recorded. The spectral widths of the indirect ¹⁵N and ¹³CO dimensions were both 2000 Hz; the spectral width of ¹H dimension was 9259.2 Hz. HNCO and HSQC data sets contained 128 (t₁)×1024 (t₂) and 192 (t₁)×1024 (t₂) complex data points, respectively. NMR data sets were processed with Felix 98.0 software.

Results

We adapted the fermentation procedure of the yeast Pichia pastoris to grow it on ¹³C/¹⁵N-isotope enriched medium for labeling of the AVR4 protein, as fermentors offer the highest yield due to an optimal growth rate. So far, ¹⁵N-labeling in P. pastoris has largely been restricted to shake flask cultures and for a large number of overexpressed proteins ¹³C-labeling is prohibitively expensive in shake flask cultures due to the low protein yields. Wood and Komives (1999) showed that ¹⁵N-labeling in a fermentor is more cost effective than in shake flask cultures but that ¹³C-labeling in a fermentor using a Mut^{S} strain is expensive, mainly due to the large minimal volume needed in their fermentation vessel (see also below). Here we show that it is economically feasible to perform a double ¹³C/¹⁵N-labeling of proteins in a fermentor using the Mut⁺ strain of P. pastoris.

Ammonium Sulfate as ¹⁵N-nitrogen Source and pH Control with Potassium Hydroxide

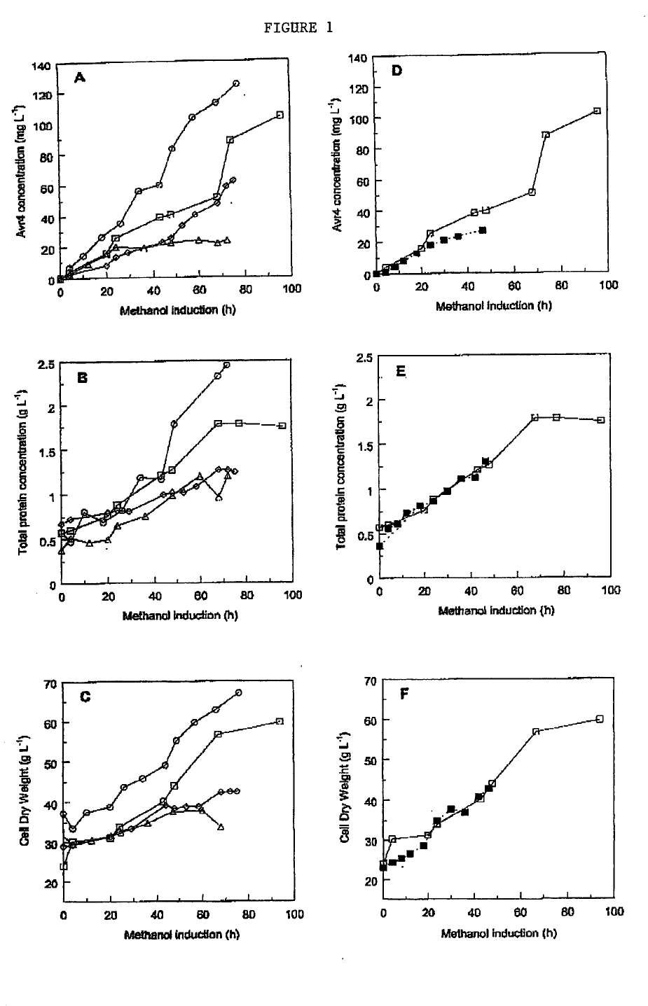

¹⁵N-isotope incorporation was achieved by replacing NH₄OH by (¹⁵NH₄)₂SO₄ as nitrogen source. The constant acidification of the medium is normally compensated with NH₄OH. Instead of NH₄OH we used KOH to control the pH in the case of ¹⁵N-labeling. For P. pastoris the supplied amount of (NH₄)₂SO₄ had been shown to influence protein expression levels substantially (Wood and Komives, 1999), while nitrogen limitations have been reported to increase protease activity (McAlister et al., 1998). Therefore, we ascertained that (NH₄)₂SO₄ concentrations would be optimal. The effects of different (NH₄)₂SO₄ supplies on AVR4 secretion and Cell Dry Weight (CDW) were examined (FIG. 1).

In addition, the combination of KOH and (NH₄)₂SO₄ can cause K₂SO₄ to precipitate as salt concentrations steadily increase by the KOH supplies needed to compensate for the acidification, which eventually leads to an arrested growth. As a way to circumvent the effects of increased salt concentrations, a medium exchange was recommended prior to the methanol-induction phase for the Mut^{S} strain (Wood and Komives, 1999). In our approach no medium exchange was applied. The effects of increased salt concentrations were examined over the time course of the experiment for the different (NH₄)₂SO₄ supply regimes.

For comparison the general set-up of the fermentation contained a glycerol batch phase of 20 h over which the CDW increased to ˜24 g/L, which is in agreement with the expected growth yield on glycerol. During the glycerol fed-batch phase DO levels were kept steady at 35% for 4 h and CDW increased to ˜34 g/L.

We achieved expression levels with (NH₄)₂SO₄ that reached the levels obtained with NH₄OH if aliquots of 10 g (NH₄)₂SO₄ per liter medium were applied every 24 h (resulting in more than 100 mg AVR4 protein per liter). These levels were 83% of the levels obtained in a NH₄OH controlled fermentor (FIG. 1A) which may be classified as an intermediate yield for P. pastoris when compared to the reported yields by Cregg et al. (1993). Under these conditions AVR4 concentrations became 24 times higher in a fermentor than in flask cultures, which is in good agreement with other reports (Cregg et al., 1993).

After 24 h, the initial supply of 10 g/L (NH₄)₂SO₄ had been completely consumed as noticed by the fact that the yeast had stopped acidifying the medium. After adding 10 g/L (NH₄)₂SO₄ acidification restored again. DO levels were not seriously effected by this addition. This cycle of (NH₄)₂SO₄ supplies was repeated over the next 48 h. As CDW and the total protein content evolved comparably in both 10 g/L (NH₄)₂SO₄ and in the NH₄OH controlled fermentations, we concluded that the growth was never under nitrogen starvation conditions if 10 g/L (NH₄)₂SO₄ was supplied every 24 h (FIGS. 1B and 1C). Nitrogen limitations became evident as growth retarded as soon as less than 10 g/L (NH₄)₂SO₄ was applied every 24 h (diamond; FIG. 1C). Additionally, with a supply of 5.0 g/L the yeast stopped acidifying within 12 h after the start of the glycerol batch phase. With the addition of 5.0 g/L (NH₄)₂SO₄ after 24 h the DO levels dropped to such an extent that the methanol supply was not started until the DO levels had restored again. Proteolytic degradation of AVR4 was not observed under these conditions on SDS-PAGE. On the other hand, higher concentrations of (NH₄)₂SO₄ inhibited protein secretion (triangle, FIG. 1A). Growth lagged seriously when 20 g/L (NH₄)₂SO₄ was applied every 24 h (FIG. 1C). Also K₂SO₄ had precipitated at the end of the methanol-induction phase. However, only half the amount of nitrogen is supplied at the optimum of 10 g/L of (NH₄)₂SO₄ per 24 h compared to the NH₄OH controlled fermentor.

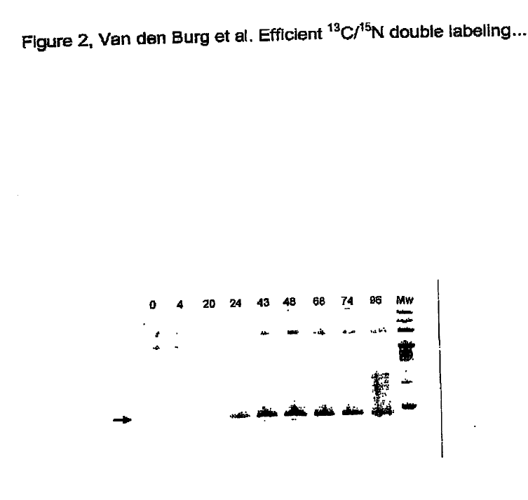

Based on the results obtained with the supply of 10 g/L (NH₄)₂SO₄ every 24 h, we concluded that no medium exchange was needed. The absence of salt effects on the growth could be explained by the fact that the first two phases only lasted for 24 h. In the approach with a medium exchange the first two phases lasted for 60 h prior to the medium exchange (Wood an Komives, 1999). Therefore, elevated salt concentrations could only affect growth at the end of the methanol-induction phase in our case. Indeed, 72 h after the start of the methanol-induction phase growth lagged in our approach and other proteins showed up in the medium as seen by SDS-PAGE (FIG. 2). The acidification did not resume after the third (NH₄)₂SO₄ pulse at 72 h. The fermentation was stopped at 96 h (FIG. 1).

¹³C/¹⁵N Labeling of the AVR4 Protein

With the optimal (NH₄)₂SO₄ conditions AVR4 levels reached 40 mg/L within 48 h of the methanol-induction phase which was sufficient for cost effective ¹³C-labeling. We, therefore, decided to reduce the fermentation volume to save on costs for ¹³C-labeling. A volume of 300 mL would provide enough ¹³C/¹⁵N-labeled AVR4. A yield of 30 mg/L of AVR4 was obtained in this small vessel (FIG. 1D). CDW reached 43.5 g/L, which is slightly less than in the larger vessel (FIG. 1F). This small decrease in CDW was partially caused by a shorter glycerol fed-batch phase from 4 h to only 30 min, which was sufficient to derepress the AOX1 promoter. The total amount ¹³C₃-glycerol used was hereby reduced with 50%. Only 15 g ¹³C₃-glycerol and 50 g ¹³C-methanol were needed (Table 1). Additionally, we diluted the methanol supply to circumvent toxicity. At the end of the fermentation the total fermentation volume had increased by 200 mL which prevented serious salt accumulation, but also decreased CDW.

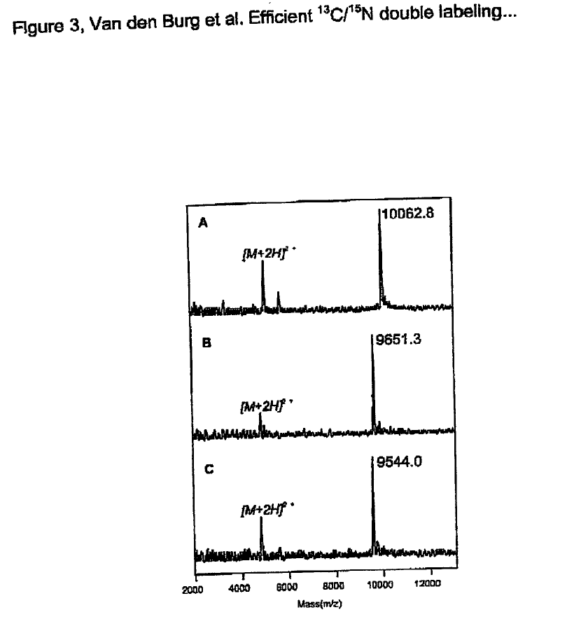

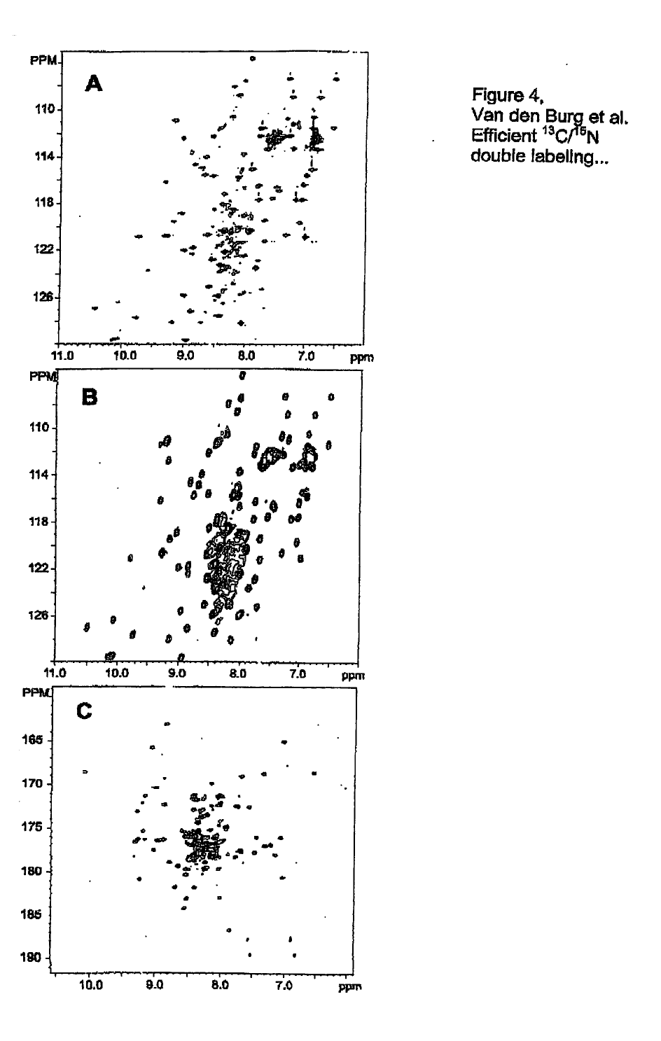

The AVR4 protein was labeled with an efficiency of +98% in ¹³C- and ¹⁵N-isotopes as shown by mass spectrometry (FIG. 3). All eight Cys residues were shown to be involved in disulfide bonds and no glycosylation had occurred as expected. The heterologous AVR4 protein behaved similar to the native AVR4 produced by C. fulvum. A hypersensitive response was specifically induced on Moneymaker Cf4 tomato plants upon injection of the heterologous AVR4 protein into the leaves and an antibody raised against native AVR4 recognized the heterologous AVR4 protein (results not shown). Structural integrity was accessed by a ¹H-¹⁵N HSQC spectrum on both ¹⁵N- and ¹³C/¹⁵N-labeled AVR4 samples (FIGS. 4A and 4B). Both line width and chemical shift dispersion were consistent with a substantially folded protein. The sample has been highly stable for months now and triple resonance experiments like a HNCO experiment (FIG. 4C) were performed. Detailed structural analysis and dynamic studies are in progress and shall be reported elsewhere.

Discussion

Overexpression and efficient ¹³C/¹⁵N-labeling of any protein is essential in order to study its structure-function relationship by NMR. However, overexpression turned out not to be easy for the avirulence protein AVR4 of Cladosporium fulvum as Escherichia coli, the fungus Aspergillus niger, and the fungus C. fulvum itself failed to overexpress AVR4 in spite of serious attempts. To overexpress AVR4, we, therefore, employed the methylotrophic yeast Pichia pastoris as it handles disulfide bonds very well, it does not hyperglycosylate heterologous proteins which frequently occurs with the yeast Saccharomyces cerevisiae (Montesino et al., 1998), and secretion required for proper folding of the AVR4 protein is easily achieved at high protein yields. Using P. pastoris we achieved yields of 126 mg/L of AVR4 protein in a fermentor, while in batch flask culture yields remained below 5 mg/L. These expression levels are not excessively high as more than 10 g heterologous protein per liter has been reported for P. pastoris fermentations (Cregg et al, 1993; Laroche et al, 1993). In spite of this limited expression level of AVR4 we succeeded in ¹³C/¹⁵N-labeling of AVR4 at relatively low costs. We expect that this report will advertise a wider use of P. pastoris for overexpression of ¹³C/¹⁵N-labeled proteins.

From our studies we conclude that three factors are important for efficient ¹³C/¹⁵N labeling in P. pastoris: (1) the use of a small fermentor vessel, (2) the length of the glycerol fed-batch, and most importantly (3) the use of the Mut⁺ strain in combination with the first two factors.

Choice of the Strain: Mut⁺ or Mut^{S}

The choice of the strain was determined by the optimal feed rates, glycerol consumption in the glycerol fed-batch phase and methanol-induction phase (i.e. the second and third phase), and the prices of the carbon sources. The growth rate in the methanol-induction phase positively influences the protein secretion levels (d'Anjou and Daugulis, 1997). On methanol the Mut⁺ strain grows at a rate of 0.14 h⁻¹, while the Mut^{S} strain only grows at a rate of 0.035 h⁻¹ (Brierley et al., 1990). Therefore, the methanol-induction phase will take longer for the Mut^{S} strain (175 h) than for the Mut⁺ strain (45 h) to reach the same expression levels. And although the methanol feed rate of the Mut^{S} strain will be half the rate of the Mut⁺ strain, more ¹³C-methanol will still be needed for the Mut^{S} strain. The growth rate of the Mut^{S} strain can be increased to 0.14 h⁻¹ by a combined feed of glycerol and methanol with an optimal glycerol feed rate of 2.0 g·L⁻¹h⁻¹ and a glycerol:methanol ratio of 2:1 (v/v) (Egli et al., 1986; Brierley et al., 1990). Based on the current market price of ¹³C₃-glycerol, which is 3.5 times more expensive than ¹³C-methanol (per gram), we concluded that ¹³C-incorperation is two to four times more expensive with the Mut^{S} strain than with the Mut⁺ strain.

In table 1, we compare our fermentation protocol using the Mut⁺ strain with previous reports in which also (NH₄)₂SO₄ is applied for ¹⁵N-labeling in a fermentator. As can be seen from table 1 our efforts result in a cost reduction for ¹³C-labeling. ¹³C-Labeling using the Mut^{S} strain would be at least two times more expensive than with the Mut⁺ strain (per volume). In particular this difference can be ascribed to a more efficient biomass accumulation with Mut⁺ strain. Another major factor in the cost reduction is the decreased fermentation volume. Hitherto, ¹³C-labeling had been restricted to flasks cultures although the yields are limited in flasks. The lower limit of the fermentation volume (˜1 L up to now) has largely restricted its use for ¹³C-labeling. It is emphasized that the efficiency of the fermentations presented in table 1 can not be estimated by their respective protein yields, as those have been largely influenced by the heterologous gene itself and the copy number of the heterologous gene (Clare et al, 1991; Cregg et al., 1993). However, carbon and nitrogen consumption may be compared for the different protocols as the total consumption is independent for the method by which the strain was obtained i.e. site of chromosomal integration of the gene (i.e. AOX1 or HIS4 loci) or type of integration (insertion or transplacement) (Clare et al., 1991).

Length of the Glycerol Fed-batch Phase (Second Phase)

By shortening the glycerol fed-batch phase from 4 h to only 30 min, costs for ¹³C-labeling were substantially reduced. In most cases this short period in combination with a methanol-induction phase of 48 h will be sufficient as the heterologous protein will have accumulated to the desired levels. If necessary, the methanol-induction phase can be extended for another 24 h without negative effects (during this period AVR4 levels increased to almost 50 mg/L). On the other hand, the role of this second phase is considered twofold, (1) the AOX1 promoter is derepressed and (2) the biomass is substantially increased over 4 h. Wood and Komives (1999) suggested even an extension of the glycerol fed-batch phase from 4 to more than 24 h for the Mut^{S} strain which guaranteed cell densities at the start of the induction which reached the level normally obtained at the end of fermentation, but this extension required 100 g/L of glycerol which would raise costs for ¹³C-labeling excessively (Table 1).

The Optimal (NH₄)₂SO₄ Supply

We determined an optimal (NH₄)₂SO₄ supply for the Mut⁺ strain of 10 gram per liter every 24 h. The nitrogen limitations effected the protein yields clearly as the yield of AVR4 improved with 30% as the (NH₄)₂SO₄ supply increased from 5 g to 10 g/L every 24 h. This increase is less pronounced than reported by Wood and Komives (1999). If one corrects for the different (NH₄)₂SO₄ regimes the total amount consumed is quite comparable over time for both regimes. In the regime of Wood and Komives, the discontinuous (NH₄)₂SO₄ supplies could have caused a more pronounced temporary nitrogen starvation when too little (NH₄)₂SO₄ was applied. Considering this negative effect on the protein yields, we conclude that the addition of (NH₄)₂SO₄ every 24 h is preferred.

Besides an arrested acidification, a steady increase of the DO levels in time also indicates growth retardation. The adaptation to the growth on methanol takes ˜4 h after which the DO levels are kept constant by a slight increase of the methanol feed rate. Even at the end of the methanol-induction phase, DO levels had never become higher than 60%. Loewen et al. (1997) described that the DO levels had reached almost 100% at the end of their fermentation (Table 1). With a rate of 10 g/L of (NH₄)₂SO₄ being consumed every 24 h as here described, the total supply of 20 g/L (NH₄)₂SO₄ would have been consumed within 48 h with its consequences. The nitrogen limitation in this case was not only reflected in the increased DO readings, but also the increase CDW lagged in the second half of the fermentation run. Again, these data strongly suggest that balancing the nitrogen supply is crucial for ¹⁵N-labeling in a fermentation.

Although a few alternatives for ¹³C-labeling could be appealing, most probably they will not be as successful as the protocol described here. ¹³C₆-Glucose as replacement of ¹³C₃-glycerol looks attractive as the growth rate remains the same (Brierley et al., 1990). However, glucose is a strong repressor of the AOX1 promoter, and the glycerol fed-batch phase would, therefore, take longer than with ¹³C₃-glycerol. The current market prices are virtually identical for ¹³C₆-glucose and ¹³C₃-glycerol, although in the past ¹³C₃-glycerol was relatively more expensive than ¹³C₆-glucose. Therefore, the use of ¹³C₆-glucose is less cost effective for ¹³C-labeling of proteins than the use of ¹³C₃-glycerol.

Although the replacement of glycerol for methanol seems to separate two different phases, namely biomass accumulation and induction of the heterologous gene, Wood and Komives (1999) showed that 70% of the carbon incorporated in the heterologous protein comes from the cell mass which was present at the start of the methanol-induction phase, which excludes the isotopic enrichment of only one of the two carbon sources. Enriched yeast extract media (which are often used with E. coli) used in batch flask cultures are not expected to improve protein yields substantially as growth of P. pastoris is limited by the aeration in the flasks. Moreover, protein purification is often easier form the salt based medium in which the heterologous protein is the predominantly secreted protein as seen on SDS-PAGE (Penheiter et al., 1998).

In conclusion, cost-effective ¹³C/¹⁵N-labeling for triple resonance experiments with our protocol may be useful for the production of many other proteins with difficult folding pathways. Interestingly, deuterium labeling was achieved using P. pastoris without deuterated carbon sources, but solely with D₂O (Massou et al., 1999; Morgan et al., 2000). Deuterium labeling could also be performed with our approach using the Mut⁺ strain. Thus, we have shown that, P. pastoris is an attractive alternative whenever E. coli is unsuitable.

The disclosures of the following references are hereby incorporated by reference.

References

1. Brierley, R. A., Bussineau, C., Kosson, R., Melton, A. and Siegel, R. S. (1990), Ann. New York Acad. Sci., 589, 350-362.

2. Clare, J. J., Rayment, F. B., Ballantine, S. P., Sreekrishna, K. and Romanos, M. A. (1991), Bio/Technology, 9, 455-460.

3. Cregg, J. M., Tschopp, J. F., Stillman, C., Siegel, R., Akong, M., Craig, W. S., Buckholz, R. G., Madden, K. R., Kellaris, P. A., Davis, G. R., Smiley, B. L., Cruze, J., Torregrossa, R., Velicelebi, G., Thill, G. P. (1987), Bio/Technology, 5, 479-485.

4. Cregg, J. M., Vedvick, T. S. and Raschke, W. C. (1993), Bio/Technology, 11, 905-910.

5. d'Anjou, M. C. and Daugulis, A. J. (1997), Biotechnol. Techniq., 11, 865-868.

6. Denton, H., Smith, M., Husi, H., Uhrin, D., Barlow, P. N., Balt, C. A. and Sawyer, L. (1998), Protein Expr. Purif., 14, 97-103.

7. Digan, M. E., Lair, S. V., Brierley, R. A., Siegel, R. S., Williams, M. E., Ellis, S. B., Kellaris, P. A., Provow, S. A., Craig, W. S., Veligelebi, G., Harpold, M. M. and Thill, G. P. (1989), Bio/Technology, 7 160-164.

8. Egli, T., Bosshard, C. and Hamer, G. (1986), Biotechnol. Bioeng., 18, 1735-1741.

9. Joosten, M. H. A. J., Cozijnsen, T. J. and De Wit, P. J. G. M. (1994), Nature, 367, 384-386.

10. Joosten, M. H. A. J., Vogelsang, R., Cozijnsen, T. J., Verberne, M. C. and De Wit, P. J. G. M. (1997), Plant Cell, 9, 367-379.

11. Karas, M. and Hillenkamp, F. (1988), Anal. Chem., 60, 2299-301.

12. Kay, L. E., Keifer, P. and Saarinen, T. (1992), J. Am. Chem. Soc., 114, 10663-10665.

13. Kussmann, M., Nordhoff, E., Rahbek-Nielsen, H., Haebel, S., Rossel-Larsen, M., Jakobsen, L., Gobom, J., Mirgorodskaya, E., Kroll-Kristensen, A., Palm, L. and Roepstorff, P. (1997), J. Mass Spec., 32, 593-601.

14. Laroche, Y., Storme, V., De Meutter, J., Messens, J. and Lauwereys, M. (1994), Bio/Technology, 12, 1119-1124.

15. Loewen, M. C., Liu, X., Davies, P. L. and Daugulis, A. J. (1997), Appl. Microbiol. Biotech., 48, 480-486.

16. Markley, J. L., Bax, A., Arata, Y., Hilbers, C. W., Kaptein, R., Sykes, B. D., Wright, P. E. and Wuethrich, K. (1998), Eur. J. Biochem., 256, 1-15.

17. Massou, S., Puech, V., Talmont, F., Demange, P., Lindley, N. D., Tropis, M. and Milon, A. (1999), J. Biomol. NMR, 14, 231-239.

18. McAlister, M. S. B., Davis, B., Pfuhl, M. and Driscoll, P. C. (1998), Protein Eng., 11, 847-853.

19. Mine, S., Ueda, T., Hashimoto, Y., Tanaka, Y., Imoto, T. (1999), FEBS Lett., 448, 33-37.

20. Montesino, R., Garcia, R., Quintero, O. and Cremata, J. A. (1998), Protein Exp. Purif., 14, 197-207.

21. Morgan, W. D., Birdsall, B., Frenkiel, T. A., Gradwell, M. G., Burghaus, P. A., Syed, S. E. H., Uthaipibull, C., Holder, A. A. and Feeney, J. (1999), J. Mol. Biol., 289, 113-122.

22. Morgan, W. D., Kragt, A., and Feeney, J. (2000), J. Biol. NMR, 17, 337-347.

23. Peelen, S., Wijmenga, S. S., Erbel, P. J. A., Robson, R. L., Eady, R. R. and Vervoort, J. (1996), J. Biomol. NMR, 7, 315-330.

24. Penheiter, A. R., Klucas, R. V. and Sarath, G. (1998), Protein Exp. Purif., 14, 125-130.

25. Piotto, M., Saudek, V. and Sklenar, V. (1992), J. Biomol NMR, 2, 661-665.

26. Primus, J. L., Boersma, M. G., Mandon, D., Boeren, S., Veeger, C., Weiss, R, Rietjens, I. M. (1999), J. Biol. Inorg. Chem.; 4, 274-83

27. Romanos, M. (1995), Curr. Opin. Biotechnol., 6, 527-533.

28. Schägger, H. and Von Jagow, G. (1987), Anal. Biochem., 166, 368-379.

29. Sreekrishna, K., Brankamp, R. G., Kropp, K. E., Blankenship, D. T., Tsay, J. T., Smith, P. L., Wierschke, J. D., Subramaniam, A. and Birkenberger, L. A. (1997), Gene, 190, 55-62.

30. Stonehouse, J., Clowes, R. T., Shaw, G. L., Keller, J. and Laue, E. D. (1995), J. Biomol. NMR, 5, 226-232.

31. Stratton, J., Chiruvolu, V. and Meagher, M. (1998), In Pichia Protocols (Eds., Higgins, D. R. and Cregg, J. M.), Humana Press, Totowa, N.J., pp. 107-120.

32. Tschopp, J. J., Sverlow, G., Kosson, R., Craig, W. and Grinna, L. (1987), Bio/Technology, 5, 1305-1308.

33. Wishart, D. S., Bigam, C. G., Yao, J., Abildgaard, F., Dyson, H. J., Oldfield, E., Markley, J. L. and Sykes, B. D. (1995), J. Biomol. NMR, 6, 135-140.

34. Wood, M. J. and Komives, E. A. (1999), J. Biomol. NMR, 13, 149-159.

1. A method of isotopically labeling a protein, wherein said isotopic label is ¹³C or ¹⁵N or both ¹³C and ¹⁵N, comprising expressing said protein in the Mut+ strain of Pichia pastoris, wherein said Pichia pastoris is cultured in the presence of a ¹³C carbon source or a ¹⁵N nitrogen source or both a ¹³C carbon source and a ¹⁵N nitrogen source.
